(19) United States
(12) Patent Application Publication
(10) Pub. No.: US 2010/0248332 A1
(43) Pub. Date: Sep. 30, 2010

(54) Yeast used for pizza and its production method

(75) Inventors: Xuefeng Yu, Hubei (CN); Zhihong Li, Hubei (CN); Minghua Yu, Hubei (CN); Juan Yao, Hubei (CN); Jinbao Kuang, Hubei (CN); Jinjun Shen, Hubei (CN)

Correspondence Address: THE NATH LAW GROUP, 112 South West Street, Alexandria, VA, 22314, US

(73) Assignee: Angelyeast Co Ltd, Yichang Hubei (JP)

(21) Appl. No.: 12/733,942

(22) PCT Filed: May 18, 2009
(86) PCT No.: PCT/CN2009/071828

§ 371 Date: Mar. 30, 2010

(30) Foreign Application Priority Data
Jul. 9, 2008 (CN) 200810127691.6

(51) Int. Cl. C12N 1/16

(52) U.S. Cl. 435/255.2

(57) ABSTRACT

A yeast used for pizza and its production method are provided. The yeast is dry yeast, and the amount of CO₂ produced in the dough at 30° C. during 60 minutes is 250˜450 mL CO₂/h, wherein the dough contains those ingredients as following: 280 g flour, 1.43% salt by weigh of flour, 4 g pure dry yeast and appropriate content water. The production method includes the following steps: A) one-step fermenting; B) drying: in which, after the one-step fermenting without further fermenting; before or during the drying step, adding the emulsifier. Since adapting the first seed yeast as commercial yeast, the fermenter procedure is omitted, the process is simplified, the cost is reduced, and the yeast produced meets the property requirements of the yeast used for pizza.

### TECHNICAL FIELD

The present invention relates to the yeast used for pizza and its production method.

### BACKGROUND ART

During the production process of pizza, a little yeast is needed to improve the flavor and taste of the production. It requires the yeast with dark color, low activity and better yeast taste.

The technical schemes of similar products have not yet been provided in patents or documents. Now, the yeast added in the large-scale production of pizza premix powder generally has two kinds: one is the unqualified yeast for normally making bread or mixture of the dried tailing; the other is specially produced using special strains such as beer yeast with low biogas-producing capability.

The main defects using the tailing of dried yeast as the yeast used for pizza are following: since it is not the special yeast, the stability of product quality can not be ensured, and the microorganism content in products would be high; the yeast used for pizza is produced by using special strains with low biogas-producing capability, which needs high production requirements and high cost. At present, the yeast special for pizza has not yet been reported in any patents and documents.

In the production technology of active dry yeast, the common flow is as follows: strain preservation cuvette→slant cuvette→liquid cuvette→conical flask→Carrel flask→seed tank→fed-batch culture→commercial yeast. Wherein fed-batch culture generally is the second grade or the third grade culture, the flow of typical second grade culture is as follows: seed tank→the first generation of yeast (seed yeast)→separating and washing→fermentor→the second generation of yeast (commercial yeast). The commercial yeast is washed and centrifuged, granulated and dried to get active dry yeast. The activity of yeast produced by this technology is too high to meet the requirements of the yeast used for pizza. In addition, more procedures make technology complex, difficult controlling, more fermenting stages and more chances to contaminate bacteria.

As a result, a sort of yeast is needed on the market, which can be produced by a simple technology with a low cost, furthermore, accords with the requirements to produce pizza, i.e. a yeast with dark color, low activity and better yeast flavor.

### SUMMARY OF THE INVENTION

The present invention is to provide a yeast with low activity and suitable for making pizza, and a process thereof.

According to one aspect of the present invention, a method to produce the yeast used for pizza includes the following steps:

A) one-step fermenting;

B) drying,

Wherein, after the one-step fermenting according to the said A), without further fermenting; before or during the drying step according to the said B), adding the emulsifier.

The emulsifiers comprise at least one of the following: sorbierite monostearate, sorbitan laurate, sorbitan palmitate, sorbitan stearate, sorbitan stearin, sorbitan oleate, sorbitan glycerol trioleate.

Based on 100 parts by weight dry yeast, 0˜1.5 parts by weight of the said emulsifier are added, preferred 0˜1.2 parts by weight, still preferred 0˜1 parts by weight, more preferred 0.2˜1 parts by weight, most preferred 0.5˜0.8 parts by weight, wherein excluding 0 parts by weight.

During the said B), the drying temperature is 90˜110° C.

Before drying in the said B), filtration or concentration steps are included.

Before, during or after the procedures of the said granulating and drying, adding the additive.

The additive is selected from at least one of the group consisting of Vitamin C and butyl hydroxy anisol.

Preferred before, during the procedures of the said granulating and drying, adding the emulsifier.

Based on 100 parts by weight dry yeast, the additive amount of Vitamin C is 0.3 parts by weight, the additive amount of butyl hydroxy anisol is 0.1 parts by weight.

The final dry substance of yeast is controlled at 94%-97%.

The amount of CO₂ produced in the dough at 30° C. during 60 minutes is 250˜450 mL, wherein the dough contains those ingredients as following: 280 g flour, 1.43% salt by weight of flour, 4 g pure dry yeast and appropriate content water.

Preferred the said yeast is sugarfree strains, more preferred saccharomyces cerevisiae.

### BENEFICIAL EFFECTS

(1) From drying the eugonic first seed yeast, the yeast for pizza with low activity and darker color are satisfied.
(2) Adopting the first seed yeast as commercial yeast, the fermenting procedure is omitted, whereby the process is simplified.
(3) By using the current yeast strains and the current workshops and equipments to accomplish the requirements of producing the yeast used for pizza, the cost is reduced.
(4) Because of reducing fermenting stages, the chances to contaminate bacteria are decreased remarkably, the success rate is increased.

### PREFERRED MODES OF CARRYING OUT THE INVENTION

In the present invention, except for pointing out specially, all the contents, percents, parts are by weight (mass). In the present invention, except for pointing out specially, all the components or substances rank the grade for food.

In the present invention, the term“yeast used for pizza” means the yeast suitable for making pizza.

In the present invention, the term“sugarfree activity”, also called sugarfree fermentation ability, means the amount of CO₂ produced by the dough at 30° C. during 60 minutes, wherein the dough contains the ingredients as follows: 280 g flour, 0% sugar based on the mass of flour, 1.43% salt based on the mass of flour, 9 g fresh yeast containing 4 g pure dry yeast and appropriate content water (for example 145˜150 mL). Under the circumstance of certain amount of flour, for a person skilled in the art, it is very easy to determine the appropriate content water, because based on technology standards or reference manuals, and observing the dough's state, appropriate content water can be determined.

The inventors unexpectedly found that after the one-step fermenting of sugarfree yeast, when it was directly separated and obtained yeast from the first seed yeast milk by drying, while adding the emulsifier, the obtained yeast with the emulsifier can sufficiently meet the requirements of the yeast used for pizza, i.e. low activity, darker color.

Consequently, one aspect of the present invention involves a method to produce the yeast used for pizza, including the following steps:

A) one-step fermenting;

B) drying,

Wherein, after the one-step fermenting according to the said A), without further fermenting; before or during the drying step according to the said B), adding the emulsifier.

By adopting the first seed yeast as commercial yeast, the fermenting procedure is omitted, whereby the process is simplified, the cost is reduced. Additionally, because of reducing fermenting stages, the chances to contaminate bacteria are decreased remarkably, the success rate is increased.

Preferred before, during or after the procedures of granulating and drying, adding the additive.

Preferred before, during the procedures of granulating and drying, adding the emulsifier.

The additive is selected from at least one of the group consisting of Vitamin C and butyl hydroxy anisol.

Preferred adding Vitamin C (Vc) and/or butyl hydroxy anisol (BHA) in the process. Based on 100 parts dry yeast, preferred the additive amount of Vc is 0.3 parts, preferred the additive amount of butyl hydroxy anisol (BHA) is 0.1 parts. During the drying process, adding a certain quantity of Vc and/or BHA can improve the flavor and the quality of the yeast.

The present invention unexpectedly found that sugarfree activity can be controlled by adjusting drying temperature and the additive amount of the emulsifier. The drying temperature, for example, is in the scope of 50˜200° C., further 65˜150° C., preferred 75˜130° C., further preferred 90˜110° C., more preferred 90˜100° C., most preferred 90˜95° C. Drying temperature has a certain effect on the activity of yeast. The activity of dry yeast at 90° C. generally is 50 ml CO₂/h higher than that at 110° C. Based on 100 parts dry yeast, the additive amount of the emulsifier is 0˜1.5 parts, preferred 0˜1.2 parts, still preferred 0˜1 parts, more preferred 0.2˜1 parts, most preferred 0.5˜0.8 parts.

The emulsifier is a family of surfactants having hydrophilic groups and hydrophobic groups, most of food emulsifiers are nonionic, such as all kinds of glyceryl alcohol or sucrose alcohol from aliphatic acid; the minority is anionic, such as sodium stearyl lactylate. Currently the surfactants used for active dry yeast are sorbierite monostearate, sorbitan laurate, sorbitan palmitate, sorbitan stearate, sorbitan stearin, sorbitan oleate, sorbitan glycerol trioleate etc.

In a embodiment of the present invention, the first seed yeast milk is directly separated and dried after one-step fermenting without washing. The specific procedure is: one-step fermenting in the seed tank→granulating and drying→vacuum packing. After the seed fermentation, it is separated in the separator to get yeast milk with about 20% concentration, then filtrated through vacuum drum to get yeast block with 33% yeast dry substances, agitated and pressed in the granulator, then squashed in the screen with about 0.5 mm bore diameter, the yeast becomes strips of dragon whiskers noodles, and then goes into a drying bed and becomes granular dry yeast under the blowing of dry hot air constantly, and then takes the dry yeast out when dry substances reach 95% or so. The dry yeast is stored by vacuum packing.

The methods and conditions of one-step fermenting in a seed tank are well known in the art such as the production of high-sugar saccharomyces cerevisiae, the production of saccharomyces cerevisiae, the production of forage yeast etc. There are almost no differences among them for the fermenting conditions and methods on the stage of one-step fermenting. All the aims are to accumulate enough yeast strains in logarithm growth period.

In another preferred embodiment of the present invention, the first seed yeast milk is directly separated and dried after one-step fermenting without washing. The specific procedure is: sugarfree yeast seed→filtrated through drum→adding the emulsifier, Vc and/or BHA→granulating and drying→vacuum packing. After the sugarfree seed fermentation, the yeast milk with about 20% yeast is separated by the centrifuge, then filtrated through vacuum drum to get yeast block with 33% yeast dry substances, adding a certain quantity of the emulsifier, Vc and/or BHA, mixed by agitating and pressed in the granulator, then squashed in the screen with about 0.5 mm bore diameter, the result yeast becomes strips of dragon whiskers noodles, and then goes into a drying bed and becomes granular dry yeast under the blowing of dry hot air constantly, and then takes the yeast out when dry substances reach 95% or so, and finally the dry yeast is stored by vacuum packing.

The embodiment of the present invention is only a demonstration. Actually, of course, the step of drying can also be omitted, while directly obtaining fresh yeast. When considering the problems such as storage, package and transport etc, the step of granulating and drying is perfect to carry out.

The yeast or yeast composition produced by the method provided in the present invention meets the requirements of the yeast used for pizza, sugarfree activity is about 250˜450 mL CO₂/h.

Perfectly, the yeast in the present invention is sugarfree strains, one of saccharomyces cerevisiae (saccharomyces cerevisiae is a beer yeast strain according to the classification of family of yeast based on their use). Some saccharomyces cerevisiae strains, such as a wine dry yeast, a beer dry yeast, can ferment to produce CO₂, meanwhile the biogas-producing capability is not high, which should meet requirements of pizza's fermentation, but whose biogas-producing capability is too low, the production is not easy to control, meanwhile the preservation rate is not high, therefore, the economical practicability as the special yeast used to make pizza is not high.

The method provided in the present invention can totally use the normal current equipment, without refitting or redesigning, therefore the cost is further reduce as a result.

According to one aspect of the present invention, the sugarfree activity of the yeast used for pizza is about 250˜450 mL CO₂/h.

According to another aspect of the present invention, a yeast composition used for pizza includes the yeast described in the present invention.

The yeast compositions used for pizza in the present invention include 0.3% Vc and 0.1% butyl hydroxy anisol (BHA), additionally including 0˜1% the emulsifier.

From drying the eugonic first seed yeast, the yeast for pizza with low activity and darker color are satisfied.

By Using saccharomyces cerevisiae strains and current workshops and equipments, the cost for producing the yeast used for pizza is reduced.

### EXAMPLES

### Example 1

After sugarfree yeast seed fermentation, it is separated and concentrated using a separator, and then put into a tank and cooled to 4˜8° C. with ice water without washing, filtrated through vacuum drum to get yeast block with 33% yeast dry substances. 0.8% the emulsifier is added in into the granulator according to the weight of absolute dry yeast without adding Vc and BHA, the yeast block and the emulsifier are further mixed and pressed to granulate, dried in fluidized bed, with the drying inlet air temperature at 90° C. The mixture is taken out when dry substances reach 95% or so, conveyed to the package workshop, vacuum packed.

### Example 2

After sugarfree yeast seed fermentation, it is separated and concentrated using a separator, and then put into a tank and cooled to 4˜8° C. with ice water without washing, filtrated through vacuum drum to get yeast block with 33% yeast dry substances. 0.5% the emulsifier, 0.3% Vc and 0.1% BHA are added into the granulator according to the weight of absolute dry yeast. The yeast block, the emulsifier, Vc and BHA are mixed and pressed to granulate, dried in fluidized bed, with the drying inlet air temperature at 110° C. The mixture is taken out when dry substances reach 95% or so, conveyed to the package workshop, vacuum packed.

### Example 3

After sugarfree yeast seed fermentation, it is separated and concentrated using a separator, and then put into a tank and cooled to 4˜8° C. with ice water without washing, filtrated through vacuum drum to get yeast block with 33% yeast dry substances. 1% the emulsifier, 0.3% Vc and 0.1% BHA are added into the granulator according to absolute dry yeast. The yeast block, the emulsifier, Vc and BHA are mixed and pressed to granulate, dried in fluidized bed, with the drying inlet air temperature at 90° C. The mixture is taken out when dry substances reach 95% or so, conveyed to the package workshop, vacuum packed.

### Control Example 1

The one-step fermentation of sugarfree yeast seed lasts for 26 h, and put into a tank when yeast grew flushes and accumulating certain quantity of yeast (about 180 g/L), and then centrifuged by a separator, washed once. The result is put into a yeast milk storage tank, cooled to 4˜8° C. with ice water for application. And then, commercial yeast fermentation is conducted. The fermenting time of commercial yeast is about 15 h. Due to inoculating with a large quantity of yeast milk in logarithm growth period, yeast growth rate is becoming higher. The cells of the yeast will mature by controllable technology in the later stage, and thus to reduce budding propagation, and increase the storage substances in the cell as to remain cell activity during the course of drying storage and rehydration. Generally the wet weight of commercial yeast can reach 240 g/L, and the yeast is put into the tank, later centrifuged and washed twice, then filtrated by vacuum drum, adding certain quantity of the emulsifier (generally 1.2% based on absolute dry yeast), granulated and dried to get dry yeast with high activity.

### Control Example 2

After sugarfree yeast seed fermentation, it is separated and concentrated using a separator, and put into a tank and cooled to 4˜8° C. with ice water without washing, filtrated through vacuum drum to get yeast block with 33% yeast dry substances, and then without any emulsifier, pressed to granulate by a granulator, dried in fluidized bed, with the drying inlet air temperature at 90° C., and then taken out when dry substances reach 95% or so, conveyed to the package workshop, vacuum packed.

### Property Test

the fermenting ability of yeast is shown in Table 1 based on the amount (mL) of CO₂ produced in the dough formulated at 30° C. during 60 minutes.

TABLE 1
The formulation of the dough used to determine the fermenting ability.

| | The dough | |
|---|---|---|
| | containing | |
| | 0% sugar | |
| flour | 280 | g |
| sugar | 0 | g |
| salt | 4 | g |
| yeast | 4 | g |
| butter | 0 | g |
| water | 148 | mL |

Note: in this table, the weight of yeast is calculated by dry weight, i.e. absolute dry weigh of pure yeast.

The dough formulated according to Table 2 is used to make pizza.

TABLE 2
the formulation of the dough used to make pizza

| | Weight percent |
|---|---|
| | of the dough |
| flour | 100% |
| Sugar | 3% |
| salt | 1.8% |
| yeast | 1% |
| White oil | 5% |
| water | 60% |

The yeast acquired from above examples and control examples is used to make dough according to the formulation in Table 2, and then the dough is used to make pizza. The condition of making pizza was as follows: kneading the dough according the formulation in Table 2 till the dough's surface was smooth, non-handsticky. The dough is subject to proofing for 30 min (30° C., relative humidity 75%) in a proofing box, then rolls it into several pieces of circle cake in appropriate sizes, and put them into a proofing box to proof till the dough swelled properly (30° C., relative humidity 75%). The cake is taken out and put into a baking plate painting with edible oil, while making pores in the dough and covering with cheese and all kinds of furnish, and then put the cake into an oven to bake 10 min at 300° C.

20 testing professionals are organized to evaluate the appearance, the taste and the like of pizza, the results are showed in Table 3 respectively. The baking conditions in every examples and control examples are strictly controllable to ensure that the baking condition in all the test samples are totally same and thus avoiding different taste due to the different baking condition.

TABLE 3
Comparison between the yeast in the present invention and the current yeast

| | Sugarfree activity | Dough's expansion | Appearance of section of | |
|---|---|---|---|---|
| | (mL CO₂/h) | degree | Pizza | Taste of Pizza |
| Example 1 | 280 | medium | Crisper outer layer, soft inner | Better |
| | | | layer, common dough's | |
| | | | structure | |
| Example 2 | 260 | medium | Crisper outer layer, soft | Excellent |
| | | | interior, good structure with | |
| | | | elasticity | |
| Example 3 | 450 | medium | Crisper outer layer, soft | Excellent |
| | | | interior, better structure with | |
| | | | elasticity | |
| Control example 1 | 900 | Too much | Over-crisp outer layer with | ordinary |
| | | | darker, fluffy interior, too soft | |
| Control example 2 | 80 | Bad | Tight surface without | ordinary |
| | | | elasticity | |

The data in Table 3 show that the sugarfree activity of yeast is lower but proper to meet the requirements of the yeast used for pizza. While the sugarfree activity of yeast produced by traditional methods with multi-stage fermentation and drying step was too high to meet the requirements of the yeast used for pizza. However, if the first seed yeast is only dried without the emulsifier, the activity was too low to meet the requirements of the yeast used for pizza. Additionally, the taste of yeast provided in the present invention was also more excellent than that of yeast produced by traditional methods.

Of course, the present invention still has other varieties and modifications. What is said above is only the preferred examples, not aimed to define the scope of protection of the invention; Under the circumstance of not deviating from the spirit of the present invention and its essential, any changes and alternatives made by persons skilled in the art according to the specification of the present invention will be under the scope of protection claimed in the present invention.

### INDUSTRIAL PRACTICABILITY

The yeast used for pizza of the present invention uses the first seed yeast as commercial yeast, Therefore the process for making the yeast is reduced. For example, the procedure using fermenter is omitted, therefore, the cost is reduced while the produced yeast meets the property requirements of the yeast used for pizza.

1. A process to produce the yeast used for pizza, is characterized by comprising the following steps:
A) one-step fermenting;
B) drying;
Wherein, after the one-step fermentation according to the said A), without further fermenting; before or during the drying step according to the said B), adding the emulsifier.

2. The process according to claim 1, is characterized in that the said emulsifier comprises at least one of the following compounds: sorbierite monostearate, sorbitan laurate, sorbitan palmitate, sorbitan stearate, sorbitan stearin, sorbitan oleate and sorbitan glycerol trioleate.

3. The process according to claim 1, is characterized in that, based on 100 parts by weight of dry yeast, 0˜1.5 parts by weight of the said emulsifier is added as an additive, preferred 0˜1.2 parts by weight, still preferred 0˜1 parts by weight, more preferred 0.2˜1 parts by weight, most preferred 0.5˜0.8 parts by weight, wherein excluding 0.

4. The process according to claim 1, is characterized in that the dry temperature is 90˜110° C. in the said B).

5. The process according to claim 1, is characterized by further comprising filtration or concentration steps before the said B).

6. The process according to claim 1, is characterized by before, during or after the said drying procedure, adding the additive.

7. The process according to claim 6, is characterized in that the said additive is selected from at least one of the group consisting of Vitamin C and butyl hydroxy anisol.

8. The process according to claim 7, is characterized in that based on 100 parts by weight of dry yeast, 0.3 parts by weight of Vitamin C is added as an additive, and 0.1 amounts by weight of butyl hydroxy anisol is added an another addictive.

9. The process according to claim 1, is characterized by controlling the final dry substance of yeast at 94%-97%.

10. The process according to claim 1, is characterized in that the amount of CO₂ produced in the dough at 30° C. during 60 minutes is 250˜450 mL CO₂/h, wherein the dough contains those ingredients as following: 280 g flour, 1.43% salt by weigh of flour, 4 g pure dry yeast and appropriate content water.

11. The process according to claim 1, is characterized in that the said yeast is sugarfree strains, preferred saccharomyces cerevisiae.

12. A saccharomyces cerevisiae, is characterized in that it is produced by the process according to claim 1.
